(19) United States
(12) Plant Patent Application Publication
(10) Pub. No.: US 2002/0174466 P1
(43) Pub. Date: Nov. 21, 2002

(54) Sanvitalia plant named Little Sun

(75) Inventor: Michael Helmet Unger, Rheinfelden (DE)

Correspondence Address: ROCKEY, MILNAMOW & KATZ, LTD., Two Prudential Plaza, Suite 4700, 180 North Stetson, Chicago, IL, 60601, US

(73) Assignee: Unger Breeding

(21) Appl. No.: 09/819,495

(22) Filed: Mar. 28, 2001

(51) Int. Cl. A01H 5/00

(52) U.S. Cl. PLT/263

(57) ABSTRACT

A new and distinct Sanvitalia plant botanically known as Sanvitalia speciosa named Little Sun, characterized by its bright yellow flowers, mounded trailing habit, and dark green leaves.

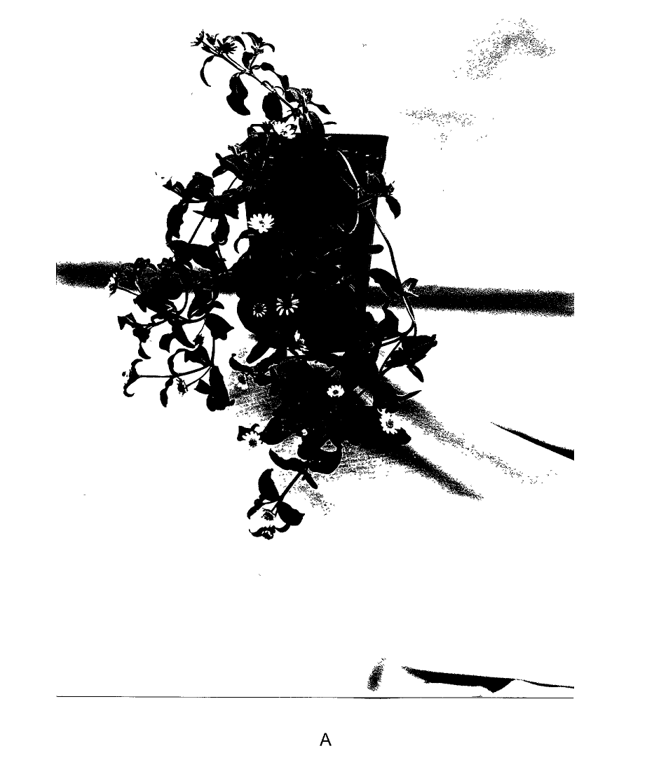

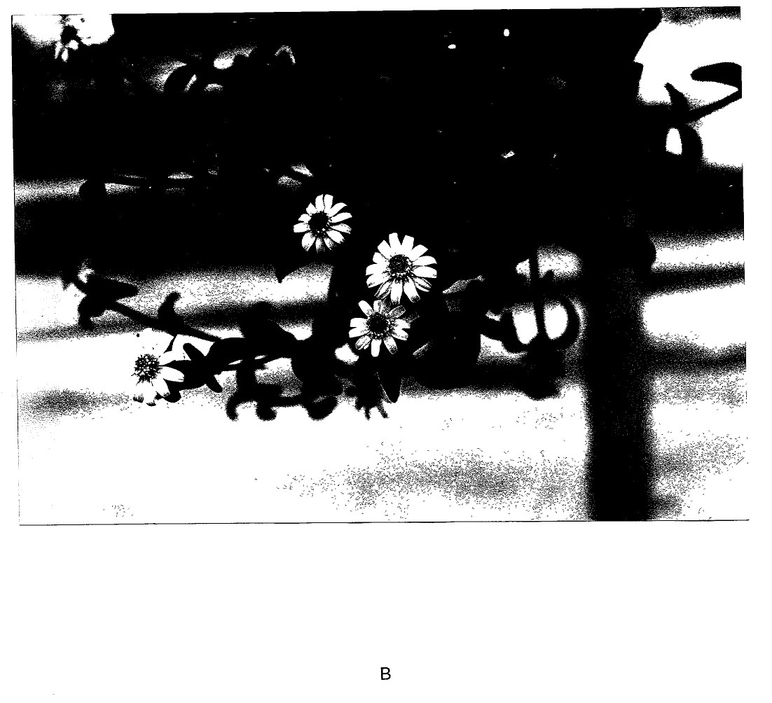

### BACKGROUND OF THE INVENTION

The present invention comprises a new and distinct Sanvitalia plant, botanically known as Sanvitalia hybrida, and hereinafter referred to by the cultivar name Little Sun.

The new cultivar was developed by the inventor through a controlled breeding program during 1996, at Rheinfelden, Germany.

The female (seed) parent of Little Sun was an unnamed proprietary Sanvitalia procumbens cultivar. The male (pollen) parent of Little Sun was the commercially available Sanvitalia aztekengold.

Asexual reproduction of the new cultivar by terminal cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits bright yellow flowers,

(b) forms medium green foliage,

(c) exhibits a good basal branching character and

(d) exhibits a mounded and trailing growth habit.

When the new cultivar of the present invention is compared to Sanvitalia aztekengold (not patented) it is found that the new cultivar has fewer branches and a more trailing habit as detailed in TABLE A.

### BRIEF DESCRIPTION OF THE PHOTOGRAPHS

The accompanying photographs show as nearly true as it is reasonably possible to make the same in color illustrations of this type, typical flower and foliage characteristics of the new cultivar. The plants were grown for 14 weeks in a greenhouse at West Chicago, Ill., U.S.A.

Photo A illustrates the trailing growth habit of Little Sun.

Photo B is a closer view of individual flowers.

### DETAILED BOTANICAL DESCRIPTION

The Little Sun cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Feb. 15, 2001. The readings were taken between 10:00 and 11:00 a.m. under natural daylight conditions. The plants were produced from cuttings taken from stock plants and grown in a double polycarbonate covered greenhouse under conditions comparable to those used in commercial practice while utilizing a soilless growth medium and maintaining temperatures of approximately 72° F. during the day and approximately 65° F. during the night. The following descriptions and measurements were taken from plants grown for 12 weeks after the planting of rooted cuttings.

Classification:

Botanical.—Sanvitalia hybrida cultivar Little Sun

Parentage:

Female parent.—Unnamed Sanvitalia procumbens

Male Parent.—Sanvitalia atztekengold

Propagation:

Type cutting.—Terminal tip

Time to Initiate Roots.—Approximately 14-21 days with the shorter times generally being experienced in the summer and the longer times in the winter.

Root Description.—Fibrous, branching

Plant Description:

Habit of Growth.—Moderately vigorous with good basal branching. Pinching improves basal branching. A mature plant, 12 to 14 weeks after the planting of a rooted cuffing, measures approximately 6.1 cm in height, approximately 16.7 cm in diameter and approximately 31.6 cm from top of pot to bottom of foliage.

Form.—Upright mounded and trailing

Lateral Stem.—Diameter: Approximately 2 mm. Texture: Very sparse pubescence. Color: 187C. Internode length: Approximately 2 cm.

Foliage.—Shape: Ovate. Arrangement: Opposite. Simple with entire margin, acute apex and clasping base.

Texture: Rough. Upper and lower surface moderately covered with stiff hairs, especially along veins on lower surface. Size: Approximately 2.6 cm in length and approximately 1.1 cm at widest point. Color: Upper surface is 143A, lower surface is 146B. Venation: Mid-vein is 187D at base, rest of veins are 137C. Petiole: Length: 1.2 cm. Color: 187D.

Flowering Description:

Outdoor Flowering Habit.—Freely flowering, with substantially continuous blooming from spring until fall.

Inflorescence type.—Single, composite, persistent.

Bud.—Flat and round, 8 mm in diameter.

Inflorescence.—Diameter: Approximately 1.8 cm. Depth: Approximately 4 mm.

Ray florets.—Quantity: 11. Appearance: Smooth. Shape: Linear. Margin: Entire. Tip: Emarginate. Length:1.1 cm. Width: 7 mm. Color: Upper surface, fully opened—Between 9A and12A, lower surface, fully opened—7A with overlay of 141 B.

Capitulum.—Diameter: 5 mm. Depth: 2 mm.

Disc florets.—Quantity: Numerous. Length: 2.2 mm. Color: 2B.

Phyllaries.—Quantity: 5 Shape: Ovate, imbricate. Margin: Entire. Apex: Acute. Length: 5 mm. Width: 4 mm. Color: 143A.

Peduncle.—Length: Approximately 2 cm. Diameter: Approximately 1 mm Texture: Rough. Color: 187C.

Fragrance.—None

Reproductive Organs.—Androecium- on disc florets only. Pollen color: 1A. Gynoecium.—on ray and disc florets.

Seed production: Seed production has not been observed.

Disease resistance: Resistance to pathogens is not claimed.

I claim:

1. A new and distinct cultivar of Sanvitalia speciosa named Little Sun substantially as herein shown and described, which:
(a) exhibits yellow flowers,
(b) forms medium green foliage,
(c) exhibits a good basal branching character and
(d) exhibits a mounded and trailing growth habit.
TABLE A

| CHARACTERISTIC | LITTLE SUN | GOLDEN AZTEC |
|---|---|---|
| PLANT HEIGHT-CM | 6.1 cm | 6.7 cm |
| PLANT LENGTH FROM TOP OF | 31.6 | 25.2 |
| POT TO BOTTEM OF | | |
| FOLIAGE-CM | | |
| INTERNODE LENGTH-CM | 2 cm | 3.3 cm |
| # LATERAL BRANCHES | 14 | 17 |
